(19) United States
(12) Patent Application Publication
(10) Pub. No.: US 2022/0079618 A1
(43) Pub. Date: Mar. 17, 2022

(54) TROCAR STABILIZATION CLAMP

(71) Applicant: Covidien LP, Mansfield, MA (US)

(72) Inventor: Sabastian K. George, Bangalore (IN)

(21) Appl. No.: 17/405,252

(22) Filed: Aug. 18, 2021

Related U.S. Application Data
(60) Provisional application No. 63/078,459, filed on Sep. 15, 2020.

(51) Int. Cl. A61B 17/34

(52) U.S. Cl. CPC A61B 17/34; A61M 2025/024; A61B 2017/3492; A61B 17/3423

(57) ABSTRACT

A trocar stabilization clamp includes a clamp body defining opposed ends. First and second clamp heads having respective first and second clamps are coupled to respective ends of the clamp body. At least a portion of the clamp body includes a flexible body section. The trocar stabilization clamp is configured to secure two trocars in place and inhibit movement of the trocars.

### CROSS-REFERENCE TO RELATED APPLICATIONS

The present application claims the benefit of and priority to U.S. Provisional Patent Application Ser. No. 63/078,459, filed on Sep. 15, 2020, the entire content of which being hereby incorporated by reference.

### BACKGROUND

### Technical Field

This disclosure relates to surgical devices. More particularly, the disclosure relates to external trocar stabilization clamps to prevent trocar movement and displacement.

### Description of Related Art

Minimally invasive surgeries, such as endoscopies or thoracoscopies, are performed by making a small incision with a tubular trocar or cannula that serves as a passage for inserting an endoscope or surgical instrument into the body. With the trocar or cannula in place in the body wall, various instruments may be inserted through the trocar into the body cavity. One or more trocars may be used during a procedure. During the surgical procedure, the surgeon manipulates the instruments within or through the trocar, sometimes using more than one instrument, and trocar, at a time. The manipulation of an instrument by a surgeon may cause frictional forces between the instrument and the trocar in which the instrument is inserted. These frictional forces may result in movement of the trocar in an inward or outward direction within the body wall. If the trocar is not fixed in place, the proximal or distal motions of the instruments through the trocar may potentially cause the trocar to slip out of the body wall or to protrude further into the body cavity.

Trocars are generally held in place using integrated retention devices such as inflatable balloons on the inside of the body and washers outside the body. Despite the many retention systems commonly used, trocars may still become dislodged during surgery. At times, surgical aides, medical students, or nurses may be required to hold a trocar and instrument in place. A new trocar anchoring device may reduce undesirable movements of trocars that occur during surgery while also maintaining the position of multiple trocars.

### SUMMARY

The disclosure relates to a trocar stabilization clamp having a clamp body defining opposed ends. A first clamp head includes a first clamp and a second clamp head includes a second clamp. The first and second clamp heads are coupled to respective ends of the clamp body.

In aspects, at least a portion of the clamp body includes a flexible body section.

It is further disclosed that the clamp body further includes first and second rigid body sections. The first and second rigid body sections are each coupled to respective ends of the flexible body section. In aspects, the first and second clamp heads are respectively connected to opposed ends of the first and second rigid body sections.

In further aspects, the first clamp head includes a third clamp proximally positioned relative to the first clamp of the first clamp head, and the second clamp head includes a fourth clamp proximally positioned relative to the second clamp of the second clamp head.

In is disclosed that the first clamp and second clamp are each configured to snap fit and clamp onto a trocar.

In aspects, the first clamp head includes a third clamp proximally positioned relative to the first clamp of the first clamp head, and wherein the second clamp head includes a fourth clamp proximally positioned relative to the second clamp of the second clamp head.

In other aspects, the first clamp is configured to snap fit a trocar having a first diameter, and the third clamp is configured to snap fit a trocar having a second diameter that is smaller than the first diameter. It is disclosed that the second clamp is configured to snap fit a trocar having the first diameter, and the fourth clamp is configured to snap fit a trocar having the second diameter that is smaller than the first diameter.

In further aspects, the first clamp head includes a fifth clamp and the second clamp head includes a sixth clamp. The fifth and sixth clamps are positioned more proximally relative to the clamp body than the respective first, second, third and fourth clamps of the first and second clamp heads.

In aspects, the clamp body includes a deflectable portion.

In additional aspects, the first and second clamp heads include friction surfaces disposed thereon.

In further additional aspects, the friction surfaces are grip pads made from plastic, rubber, silicon, or combinations thereof.

In other aspects, the trocar stabilization clamp includes an outer shell made from rubber, silicon, or plastic.

In aspects, the first and second clamp heads include clamp head tips having inward facing curved opposed side walls configured to snap fit a trocar.

In yet other aspects, the clamp head tips each have a first set of fingers protruding inward from the opposed side walls, wherein the first set of fingers define proximal portions of the opposed side walls.

In further aspects, the clamp head tips each further include a second set of fingers protruding inward from the opposed side walls wherein the second set of fingers are proximally positioned relative to the first set of fingers. The first set of fingers and second set of fingers further define third and fourth clamps.

It is disclosed that the clamp body has an adjustable length.

This disclosure also relates to a trocar stabilization clamp including a clamp body having a flexible inner core and an outer shell. A first clamp head and a second clamp head are each configured to clamp at least one trocar. The first and second clamp heads are coupled to respective opposite end portions of the clamp body.

In aspects, the clamp body includes an inner body and an outer tube configured to slide longitudinally about a common axis relative to one another so as to allow a length of the trocar stabilization clamp to be varied.

In further aspects, the clamp body includes a lock stop for fixing the position of the outer tube relative to the inner body.

### BRIEF DESCRIPTION OF THE DRAWINGS

Aspects of a trocar stabilization clamp are disclosed herein with reference to the drawings wherein:

FIG. 1 is a perspective view of two trocar stabilization clamps in use, in accordance with aspects of this disclosure;

FIG. 2 is a side view of one of the trocar stabilization clamps of FIG. 1;

FIG. 3 is a top view of a trocar stabilization clamp of the disclosure;

FIG. 4 is a top detail view of a clamp head of the trocar stabilization clamp of FIG. 3;

FIG. 5 is a plan view of a flexible trocar stabilization clamp including a flexible stiff coil, in accordance with another embodiment of the disclosure;

FIG. 6 is a side view of a trocar stabilization clamp with an inner flexible core, in accordance with yet another embodiment of the disclosure; and

FIG. 7 is a plan view of an extendable body of a trocar stabilization clamp, in accordance with a further embodiment of the disclosure.

Further details and various aspects of this disclosure are described in more detail below with reference to the appended figures.

### DETAILED DESCRIPTION OF EMBODIMENTS

Aspects of the trocar stabilization clamp, in accordance with this disclosure, will now be described in detail with reference to the figures wherein like reference numerals identify similar or identical structural elements. However, it is to be understood that the disclosed devices are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

As used herein the term “proximal” refers to a portion closer to a center point along the length of the trocar stabilization clamp, and the term “distal” refers to a portion further from the center point along the length of the trocar stabilization clamp.

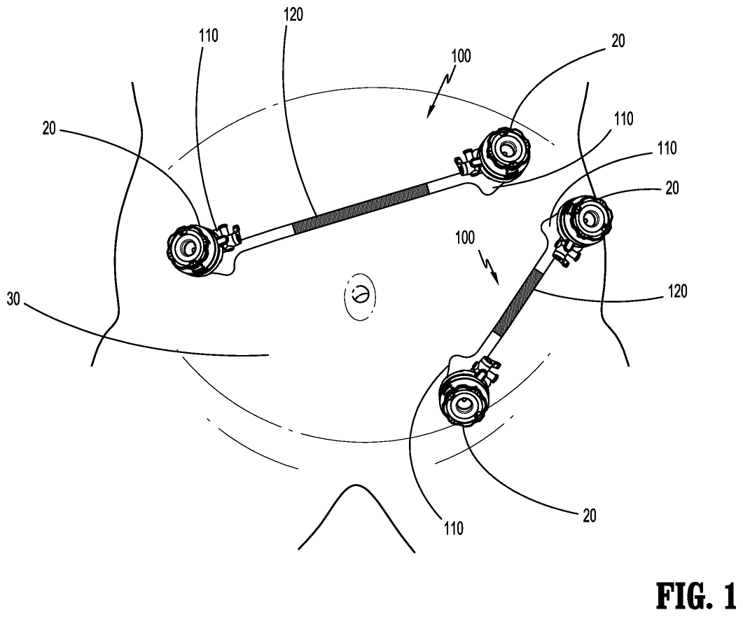

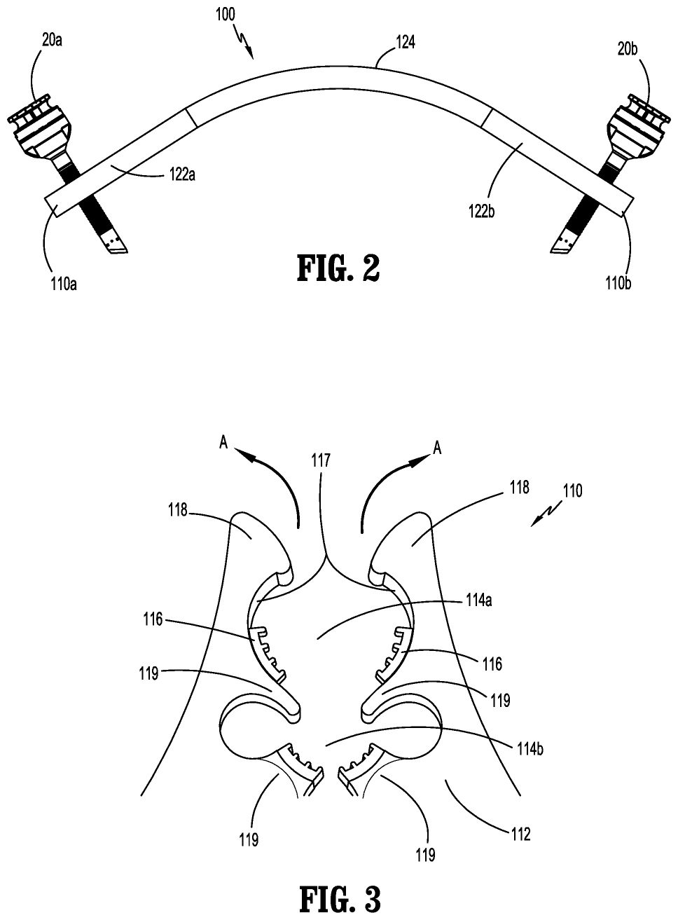

With initial reference to FIGS. 1-2, a pair of trocar stabilization clamps 100, in accordance with the disclosure, are shown respectively holding a pair of trocars 20 in place within an operative site. Each trocar stabilization clamp 100 creates tension between a pair of trocars 20 to hold the pair of trocars 20 in place, while also providing balancing forces so that movement by one or a first trocar 20a is countered by the other or a second trocar 20b thereby holding both trocars 20a, 20b in place and reducing the risk of either trocar 20a or 20b slipping further in or out of a surgical access site.

Each trocar stabilization clamp 100 includes a pair of clamp heads 110 and a clamp body 120 inter-connecting the pair or clamp heads 110.

The clamp body 120 may have various lengths, as shown in FIG. 1, for connecting trocars 20 (e.g., a first trocar 20a and a second trocar 20b) at various distances from one another. Each trocar stabilization clamp 100, and specifically, the clamp body 120 thereof, may be configured to conform to a patient's body 30 (e.g., an abdomen), such that the trocar stabilization clamp 100 may have some flexibility or curvature along the body 30. Further, the trocar stabilization clamp 100 is configured to be connected to the trocars 20a, 20b at a location adjacent to the body 30 so as not to interfere with the area surrounding the trocars 20a, 20b during surgery (FIG. 2).

Each clamp head 110 is distally coupled on opposite ends of the clamp body 120. In aspects, clamp heads 110 are symmetrically disposed along a centerline or central longitudinal axis “X-X” of the clamp body 120. In other aspects, the clamp heads 110 may be angularly coupled to the clamp body 120 and asymmetrically reflected about the centerline or central longitudinal axis “X-X” of the clamp body 120.

The pair of clamp heads 110 of a respective trocar stabilization clamp 100 are configured to clamp onto an external portion of a tubular body of a trocar or cannula 20. For example, as illustrated in FIG. 2, the pair of clamp heads 110 of a respective trocar stabilization clamp 100 includes a first clamp head 110a attached to a first trocar 20a, and a second clamp head 110b attached to a second trocar 20b.

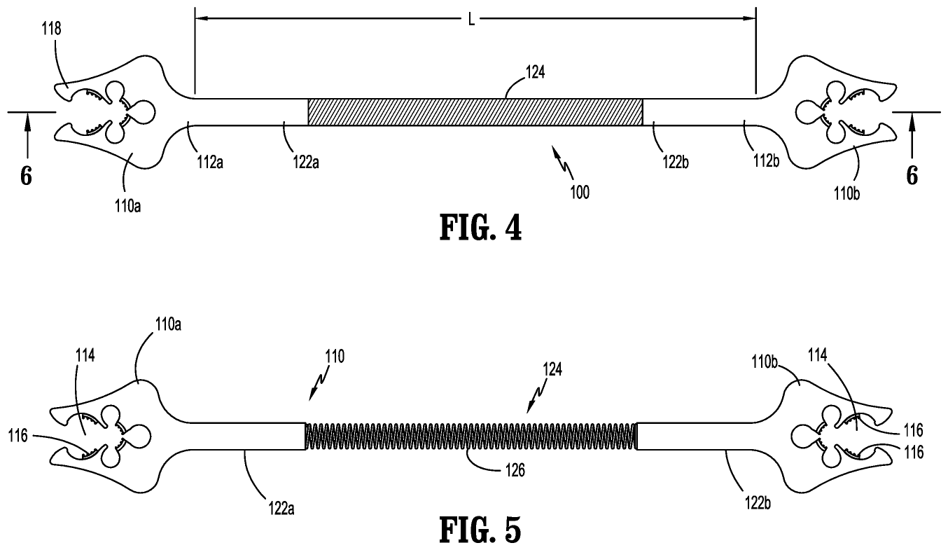

With reference to FIGS. 3-4, an exemplary clamp head 110 is shown. The clamp head 110 includes one or more clamps 114 that are configured to grasp the tubular section of the trocar 20. Each clamp head 110 may be configured to clamp trocars 20 of a single diameter or may be configured to clamp trocars 20 of varying diameters. In aspects, each clamp head 110 includes a first clamp 114a and a second clamp 114b, where second clamp 114b is proximally positioned relative to first clamp 114a. First clamps 114a are configured to grasp trocars 20 that are relatively larger in diameter while second clamps 114b are configured to grasp trocars 20 that are relatively smaller in diameter. For example, first clamps 114a may be configured to clamp a twelve (12) millimeter (mm) trocar 20 and second clamps 114b may be configured to clamp a five (5) mm trocar 20. In some aspects, the clamp heads 110 may have at least three clamps 114a, 114b, and 114c (not shown), wherein a third clamp 114c (not shown) is most proximal of the first and second clamps 114a, 114b. In this configuration, for example, first clamps 114a may be configured to clamp a fourteen (14) mm trocar 20, second clamps 114b may be configured to clamp a ten (10) mm trocar 20, and third clamps 114c (not shown) may be configured to clamp a six (6) mm trocar 20.

Each clamp head 110a, 110b may include identical sized clamps or different sized clamps. For example, first clamp head 110a may have three clamps 114 sized to grasp fourteen (14) mm, twelve (12) mm and eight (8) mm trocars 20, while second clamp head 110b may have two clamps 114 sized to grasp ten (10) mm and six (6) mm trocars 20. In other aspects, clamp heads 114 may be adjustable to accommodate trocars of various sizes.

As illustrated in FIG. 4, each clamp head includes a pair of spaced apart clamp head tips 118. The pair of clamp head tips 118 are configured to bend outwards in the direction indicated by arrows “A” so that the first clamps 114a of clamp heads 110 may readily engage a trocar 20 in a snap fit or friction fit manner. In further aspects, second clamps 114b of clamp heads 110 are similarly configured to snap fit or friction fit a smaller trocar 20 that is not large enough to be grasped by first clamps 114a. Clamps 114a, 114b may be defined by the pair of clamp head tips 118, where the pair of clamp head tips 118 protrude from the clamp body 120 and have mirror arced or concave inner facing side walls 117. To increase the clamp head tips' 118 capacity to bend and snap fit a trocar 20, the clamp head tips are configured to have fingers 119 protruding therefrom that may define first clamps 114a, second clamps 114b or portions thereof.

Clamp heads 110 may include friction surfaces 116 disposed along the pair of clamp head tips 118 for improved grip. Friction surfaces 116 may be pads made of a material selected from, but not limited to, rubber, plastic, or silicon. In aspects, the friction surfaces 116 may entirely cover an inner of side wall 117 of tips 118 of clamp heads 110.

In another aspect, clamp heads 110 may be configured to clamp to a trocar using a strap (not shown) wrapped around the trocar and secured to a receiving screw or strap securing device (e.g., a magnet or clip, not shown). In various aspects, clamp heads 110 may include scissor graspers (not shown) for clamping a trocar. Clamp heads 110 may include any construction known to those of ordinary skill in the art for grasping or clamping onto an object.

Referring now to FIG. 3 clamp body 120 defines a length “L” measured from a proximal portion 112a of first clamp head 110a to a proximal portion 112b of second clamp head 110b. The length “L” of clamp body 120 may be varied, such that the trocar stabilization clamp 100 may accommodate trocars 20 variably spaced from one another. The length “L” of clamp body 120 may allow for the trocar stabilization clamp 100 to span across opposite sides of a body 30 (see FIG. 1) or the length “L” of clamp body 120 may have a short span to allow for the trocar stabilization clamp 100 to clamp two adjacent trocars 30 (see FIG. 1). Clamp body 120 includes one or more rigid body sections 122 and a flexible body section 124 therebetween, that define the length “L” thereof. Flexible body section 124 is connected at each end to a respective first rigid body section 122a and a second rigid body section 122b. In aspects, flexible body section 124 is flexible in a concave or convex vertical direction and has minimal horizontal bending and minimal torsion along the length “L.” Flexible body section 124 may be made of a material selected from, but not limited to, rubber, silicon, elastic, and metal or plastic strips. The flexible body section 124 allows the trocar stabilization clamp 100 to conform to the surface of a body 30, as shown in FIG. 2. In use, if a surgeon places two trocars 20 on opposite sides of a body 30, the added flexibility from flexible body section 124 allows the trocar stabilization clamp 100 to stabilize the trocars 20 over a rounded or curved body 30.

Rigid body sections 122a, 122b of clamp body 120 are connected to proximal portions of the flexible body section 124. The rigid body sections 122a, 122b are connected, at distal ends thereof, to the first and second clamp heads 110a, 110b, respectively. The rigid body sections 122a, 122b prevent too much flexibility from being introduced and help keep the trocar stabilization clamp 100 under tension. Flexible body portion 124 or rigid body sections 122a, 122b may have various lengths that in sum define the length “L”. In some aspects, the clamp body 120 may only include a rigid body section 122 or a flexible body section 124 when the length “L” is sufficiently short enough that flexibility of the trocar stabilization clamp 100 is not a concern. In other aspects, the clamp body 120 may only include a rigid body section 122 when only rigid sections are required to keep two trocars 20 under tension. For example, two trocars 20 may be sufficiently adjacent each other that a single rigid body section 122 is sufficient to hold the two trocars 20 in place without needing to conform over a large body section 30. In aspects, clamp body 120 may include only a flexible body section 124 that provides tension between two trocars 20. For example, two trocars 20 at large spatially distanced positions may be connected trocar stabilization clamp 100 having a clamp body 120 with only a flexible body section 124 configured to stretch to clamp the two trocars 20.

Referring now to FIG. 5, the clamp body 120 may include a flexible body section 124 that includes a flexible coil 126 having a selected degree of stiffness and rigidity. The flexible coil 126 allows the trocar stabilization clamp 100 to be maneuvered around any items that would otherwise be in a direct path between two trocars 20 while also providing sufficient tension and distribution of forces to hold the two trocars 20 in place. Additionally, the flexible coil 126 allows the clamp heads 110 to be articulated to clamp a trocar 20 at an optimal position (e.g., orthogonal to) along the cylindrical body of the trocar 20. Flexible coil 126 may alternatively be any flexible but stiff member, such as a copper, aluminum, lead or other metal wire or coil.

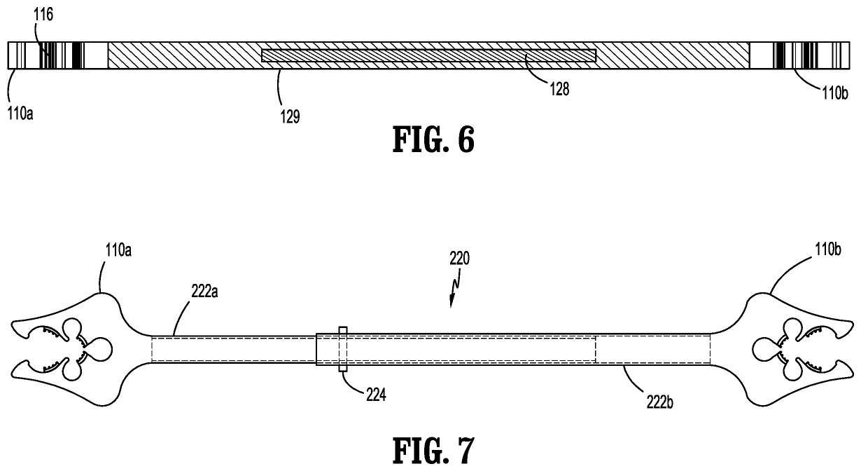

With reference to FIG. 6, the trocar stabilization clamp 100 may include an inner flexible core 128 having a selected degree of stiffness and rigidity. The inner flexible core 128 may be a thin flexible plate, strap or strip made from metal, plastic, or rubber. The inner flexible core 128 may be disposed in the clamp body 120, or it may form a core for the entire trocar stabilization clamp 100 or significant portions thereof. The inner flexible core 128 may be covered by an outer shell or sheath 129 made from a list of materials including, but not limited to, silicon, rubber, plastic, or combinations thereof. In aspects, the outer shell or sheath 129 is configured to cover the entire trocar stabilization clamp 100 to permit easier cleaning and prevent undesirable matter from becoming adhered to the trocar stabilization clamp 100. In this manner, the trocar stabilization clamp 100 may be easily cleaned and sanitized for use in another surgery. In other aspects, the outer shell or sheath 129 may include anti-microbial properties.

Referring now to FIG. 7, a trocar stabilization clamp 200, in accordance with another embodiment, is shown having an adjustable clamp body 220. The trocar stabilization clamp 200 includes clamp heads 110 supported at opposed ends of clamp body 220. The adjustable clamp body 220 has two tubular sections 222a and 222b configured to slide relative to one another. Tubular section 222a may be disposed internally of tubular section 222b, or vice versa. A lock stop 224, such as a lock collar, fixes the position of tubular section 222a with respect to tubular section 222b. In this manner, the length “L” of clamp body 220 may be adjustable such that a single trocar stabilization clamp 200 may be used to clamp trocars 20 spaced at variable distances from one another. In aspects, tubular sections 222a, 222b may be flexible and have a degree of stiffness to permit articulation about or conformity to a body 30. Tubular sections 222a, 222b are merely examples of an adjustable clamp body 220, and other constructions for a clamp body 220 having an adjustable length are well known to those of ordinary skill in the art and may be used. For example, the adjustable clamp body 220 may be an expandable flexible stiff coil 126 (FIG. 5), or one of sections 222a or 222b may be non-tubular and may be disposed within the other section 222a or 222b being tubular.

In practice, after a user has positioned two trocars 20 for surgical access in a body 30, the user may place the trocar stabilization clamp 100 to prevent movement of the trocars 20 by coupling first clamp head 110a onto a first trocar 20 and then coupling second clamp head 110b onto a second trocar 20. In aspects, a second trocar stabilization clamp 100 may additionally be coupled to the first or second trocars 20 to couple to a third trocar 20. As a surgeon maneuvers instruments within the trocar 20, potentially causing torques or forces that may dislodge the trocar 20 or move the trocar 20 inward or outward relative to body 30, the trocar stabilization clamp 100 stabilizes the trocar 20 and distributes any forces between the two clamped trocars 20 and the trocar stabilization clamp 100 that would otherwise cause the trocar 20 to become dislodged or move.

It should be understood that the foregoing description is only illustrative of the disclosure. Various alternatives and modifications can be devised by those skilled in the art without departing from the disclosure. Accordingly, the disclosure is intended to embrace all such alternatives, modifications, and variances. The embodiments described with reference to the attached drawing figures are presented only to demonstrate certain examples of the disclosure. Other elements that are insubstantially different from those described above and/or in the appended claims are also intended to be within the scope of the disclosure.

What is claimed is:

1. A trocar stabilization clamp comprising:
a clamp body defining opposed ends;
a first clamp head having a first clamp; and
a second clamp head having a second clamp;
wherein the first and second clamp heads are coupled to respective ends of the clamp body.

2. The trocar stabilization clamp of claim 1, wherein at least a portion of the clamp body includes a flexible body section.

3. The trocar stabilization clamp of claim 2, wherein the clamp body further includes first and second rigid body sections, wherein the first and second rigid body sections are each coupled to respective ends of the flexible body section, and wherein the first and second clamp heads are respectively connected to opposed ends of the first and second rigid body sections.

4. The trocar stabilization clamp of claim 3, wherein the first clamp head includes a third clamp proximally positioned relative to the first clamp of the first clamp head, and wherein the second clamp head includes a fourth clamp proximally positioned relative to the second clamp of the second clamp head.

5. The trocar stabilization clamp of claim 1, wherein the first clamp and second clamp are each configured to snap fit and clamp onto a trocar.

6. The trocar stabilization clamp of claim 1, wherein the first clamp head includes a third clamp proximally positioned relative to the first clamp of the first clamp head, and wherein the second clamp head includes a fourth clamp proximally positioned relative to the second clamp of the second clamp head.

7. The trocar stabilization clamp of claim 6, wherein the first clamp is configured to snap fit a trocar having a first diameter, and the third clamp is configured to snap fit a trocar having a second diameter that is smaller than the first diameter; and
wherein the second clamp is configured to snap fit a trocar having the first diameter, and the fourth clamp is configured to snap fit a trocar having the second diameter that is smaller than the first diameter.

8. The trocar stabilization clamp of claim 6, wherein the first clamp head includes a fifth clamp and the second clamp head includes a sixth clamp, the fifth and sixth clamps are positioned more proximally relative to the clamp body than the respective first, second, third and fourth clamps of the first and second clamp heads.

9. The trocar stabilization clamp of claim 1, wherein the clamp body includes a deflectable portion.

10. The trocar stabilization clamp of claim 1, wherein the first and second clamp heads include friction surfaces disposed thereon.

11. The trocar stabilization clamp of claim 10, wherein the friction surfaces are grip pads made from plastic, rubber, silicon, or combinations thereof.

12. The trocar stabilization clamp of claim 1, further comprising an outer shell made from rubber, silicon, or plastic.

13. The trocar stabilization clamp of claim 1, wherein the first and second clamp heads include clamp head tips having inward facing curved opposed side walls configured to snap fit a trocar.

14. The trocar stabilization clamp of claim 13, wherein the clamp head tips each have a first set of fingers protruding inward from the opposed side walls, wherein the first set of fingers define proximal portions of the opposed side walls.

15. The trocar stabilization clamp of claim 14, wherein the clamp head tips each further include a second set of fingers protruding inward from the opposed side walls wherein the second set of fingers are proximally positioned relative to the first set of fingers, wherein the first set of fingers and second set of fingers further define third and fourth clamps.

16. The trocar stabilization clamp of claim 1, wherein the clamp body has an adjustable length.

17. A trocar stabilization clamp comprising:
a clamp body including:
a flexible inner core; and
an outer shell; and
a first clamp head and a second clamp head, each clamp head configured to clamp at least one trocar, the first and second clamp heads coupled to respective opposite end portions of the clamp body.

18. The trocar stabilization clamp of claim 17, wherein the clamp body includes an inner body and an outer tube configured to slide longitudinally about a common axis relative to one another so as to allow a length of the trocar stabilization clamp to be varied.

19. The trocar stabilization clamp of claim 18, wherein the clamp body includes a lock stop for fixing the position of the outer tube relative to the inner body.
